(19) United States
(12) Patent Application Publication
(10) Pub. No.: US 2021/0202051 A1
(43) Pub. Date: Jul. 1, 2021

(54) EXTENDING PREVENTIVE HEALTH CARE INTO COMMUNITY-BASED SETTINGS

(71) Applicant: Aristotle Mannan, Weston, MA (US)

(72) Inventor: Aristotle Mannan, Weston, MA (US)

(21) Appl. No.: 16/727,835

(22) Filed: Dec. 26, 2019

(51) Int. Cl. G16H 10/60; G16H 15/00

(52) U.S. Cl. CPC G16H 10/60; G16H 15/00

(57) ABSTRACT

The present invention is generally related to a digital health platform useful in improving health care outcomes for the underserved population. More specifically, the present invention provides an inexpensive method and a system based on a digital health platform to connect “unreachable” Medicaid members, who are prone to become costlier “super-utilizer” patients, to preventive health care by their respective care management teams. The front end of the digital health platform of the present invention consists of a client management tool used at the community-based organizations. At the back end, this digital health platform links consenting Medicaid members identified at community-based organizations to their respective health care organizations for preventive health care

### CROSS-REFERENCE TO RELATED APPLICATION

This application claims the benefit of the earlier-filed U.S. Provisional Patent Application with the Ser. No. 62/784,913 filed on Dec. 26, 2018.

### STATEMENT REGARDING FEDERALLY SPONSORED RESEARCH AND DEVELOPMENT

Not Applicable.

### BACKGROUND OF THE INVENTION

When President Lyndon B. Johnson signed Medicaid into law in 1965, it was considered a small program to cover health care for the poor. Medicaid has since grown into the nation's largest health care program, covering over 75 million low-income Americans at a cost that exceeds $600 billion annually.

Medicaid must conform to federal guidelines, but can be customized at the state level through a Section 1115 waiver. Despite the uniqueness of Medicaid to each state, the programs are notorious for disproportionate consumption of taxpayer dollars. In the Commonwealth of Massachusetts alone 1.8 million people, equivalent to 25% of the state's population, receive Medicaid benefits at a cost of $17 billion per year. In the Commonwealth of Massachusetts, with an annual budget of $41 billion, Medicaid expenses account for 40% of the Commonwealth's annual budget.

Traditionally, State Medicaid programs have provided direct coverage to beneficiaries and assumed responsibility for associated costs due to medical claims. This model, however, does not allow for predictable budget forecasts and State Medicaid programs have generally struggled with patient-level care coordination. Over the past 30 years, states have outsourced risk to Managed Care Organization (MCO) health plans in the form of capitated payments that usually amount to about $6,000 per year per beneficiary. Increasingly, provider networks known as Accountable Care Organizations (ACOS) have been incentivized to improve delivery of care to the Medicaid population through value-based incentives from State Medicaid programs and MCOs.

The Medicaid population can be segmented into three groups: the “super-utilizers” which represent 5% of members, the 40% of beneficiaries who are “unreachable” and the remaining 55% who are engaged in care. The “super-utilizers” are particularly afflicted by socioeconomic risk factors including housing insecurity, food poverty as well as a general lack of support and account for 50% of expenditures, while the latter two groups account for the other 50% in costs. The disproportionate amount of resources consumed by “super-utilizers” is attributable to missed opportunities for preventive care, which ultimately manifest in costlier visits to hospital emergency departments during times of medical crisis. While “super-utilizers” are known to their respective payers and providers, approximately three quarters of this population turns over each year due to mortality. The next wave is believed to be replenished by “unreachable” members who are unengaged in care and difficult to locate due to outdated contact information.

ACOs and MCOs are penalized for a lack of screening among “unreachable” members in addition to bearing the costs associated with “super-utilizers” within their populations. It is in the best interest for ACOs and MCOs to connect “unreachable” members to preventive interventions, rather than identify individuals with the greatest needs after the fact.

Without current contact information and clinical records on the “unreachable” population, ACOs and MCOs lack a baseline to effectively deliver care. ACOs and MCOs invest heavily in outreach initiatives to ensure that their members access earlier, preventive interventions.

Multiple studies have demonstrated both clinical and financial benefits of care management for vulnerable Medicaid members. Such initiatives have been shown to reduce emergency department utilization by up to four-fold, while creating a 44% return on investment. Timing of these interventions, however, is critical given the fact that engaging an existing “super-utilizer” is less preventive and often more palliative than connecting to individuals who have yet to frequent the hospital.

Prominent strategies for member outreach to reach missing Medicaid members rely on “high tech” and “high touch” methods (FIG. 1). Companies in the high-tech realm include CareMessage, mPulse Mobile and CareSpeak Communications. These platforms aim to connect health care organizations to their Medicaid members through mobile engagement consisting of automated text messages such as reminders for prescription refills and upcoming appointments. While these “high tech” approaches certainly improve the bandwidth of health care organizations, lower income populations are prone to have greater inconsistencies with connectivity and a tendency to frequently change mobile phone numbers, thereby eliminating the channel for engagement.

The “high touch” models, also known as “feet on the street,” consist of hiring teams of individuals to conduct outreach and track down “unreachable” Medicaid members. Integra Services Connect, Advance Health and Ingenios are among the leaders in this space. In addition to navigators at ACOs and MCOs, these companies also send their staff into the community—earning revenue per member located and engaged. Health care organizations deploy outreach teams deep into neighborhoods—going door-to-door, into every alley and underneath bridges to locate vulnerable members. The costs of hiring navigators and outreach workers makes this model difficult to scale up, particularly with regards to the transient nature of the Medicaid population. Given the challenges of locating “unreachable” members through conventional, door-knocking and neighborhood canvassing, the “high touch” models have shifted towards addressing the needs of elderly Medicare members in home-based settings.

Today, engagement across the Medicaid population is unscalable and unsustainable. The market seeks a scalable as well as sustainable solution to reliably locate and engage “unreachable” Medicaid members. The present invention provides a digital health platform to more effectively locate Medicaid members and facilitate their engagement in care. The digital health platform of the present invention is referred as “bosWell”. The computer-based system of the present invention uses a unique “high-tech enabling high-touch” method to locate and engage vulnerable Medicaid members.

This present invention addresses a critical blind spot in which over a third of the 75 million low-income Americans covered by Medicaid are unengaged in care. ACO and MCO health care organizations are unable to comply with member engagement and screening benchmarks that lead to penalties as well as lower quality ratings. Furthermore, missed opportunities for preventive interventions among “unreachable” members invariably results in the creation of future “super-utilizers.” The core of bosWell, the digital health software platform according to the present invention, is a front-end client management application for community-based organizations (CBOs) that feeds into a back-end member matching and notification engine for health care organizations. As a result, “unreachable” Medicaid beneficiaries are identified in a timely manner but also provided with preventive health care.

Since CBOs are non-profit organizations, they have limited resources and lack tools for adequate recordkeeping and data reporting—often relying on pen/paper methods or homegrown spread sheets. Poor data collection compromises CBO workflows and puts these safety-net organizations at risk of becoming under-funded. A client management application component of bosWell is offered at no cost to community-based organizations (CBOs) including shelters, food pantries, resource centers and mobile clinics serving those in need. On the back-end, information gathered on consenting CBO clients is linked to their respective health care organizations in order to establish channels for engagement.

### SUMMARY OF THE INVENTION

The present invention provides a method, a system and a computer program product for identifying and engaging “unreachable” Medicaid members in preventive care in order to avoid unnecessary expensive medical treatments. The present invention leverages the fact that “unreachable” Medicaid members eligible for preventive health care visit community-based organizations (CBOs) for their immediate food, clothing, hygiene and health care needs.

In one embodiment, the present invention provides a method for identifying “unreachable” Medicaid members and providing them with preventive health care. The method for identifying “unreachable” Medicaid members starts with equipping CBOs with a client management application to capture and store longitudinal personal records of Medicaid members visiting said CBOs. The digital personal information of Medicaid members is obtained at CBOs, but cannot be disclosed to respective health care organizations without obtaining consent of Medicaid members in compliance with the Health Insurance Portability and Accountability Act of 1996 and other applicable statutes and government guidelines.

In one aspect of this method for identifying “unreachable” Medicaid members, the digital personal information of Medicaid members is transferred to a matching machine which stores the enrollment records of Medicaid members provided by ACOs and MCOs. An algorithm embedded in the matching machine queries the digital personal information of “unreachable” received from CBOs against the enrollment records already stored there to identify “unreachable” Medicaid members eligible for preventive care. Once an “unreachable” Medicaid member is located through this mechanism, an immediate alert is sent to relevant care management teams at respective ACOs and MCOs.

The matching machine is connected to both ACOs and MCOs on one side and the network of CBOs using bosWell, the digital health software platform of the present invention, on the other side through interne. The enrollment data as well CBO data stored in the matching machine is automatically updated either periodically or continuously.

In another embodiment of the present invention, a system for identifying “unreachable” Medicaid members is provided. The system according to the present invention, at the front end, has a plurality of electronic devices with client management software applications distributed at the CBOs. The staff members at the CBOs make use of this application to collect longitudinal personal data from Medicaid members visiting those CBOs. At the back end, the system for identifying “unreachable” Medicaid members has a matching engine. In one aspect of the present invention, the system for identifying “unreachable” Medicaid members has a matching engine which is already loaded with enrollment data for Medicaid members received from ACOs and MCOs. Also embedded in the matching engine is a matching algorithm for querying the digital personal records generated at CBOs using the front-end bosWell, the digital health software platform of the present invention, against ACO and MCO enrollment data present in the matching engine and identifying “unreachable” Medicaid members in need of preventive health care.

In another embodiment of the present invention, a computer program product embodied on a computer readable storage medium for processing digital personal records to identify “unreachable” Medicaid members is provided. This computer program product comprises four major components. The first component of the computer program product is a computer code for receiving personal records of “unreachable” Medicaid members visiting CBOs and generating a longitudinal digital personal records of said “unreachable” Medicaid members. This computer code is embedded in the electronic devices used by the staff at CBOs to collect personal data from the visiting Medicaid members. The second component of the computer program product is a computer code for receiving and storing digital enrollment files of Medicaid members belonging to ACOs and MCOs. The third component of the computer program product is an algorithm for matching the digital personal and records obtained using the computer code in the first component with electronic enrollment records in the second component for the purpose of identifying unreachable Medicaid members. The fourth component of the computer program product of the present invention is an electronic means to alert care management teams at ACOs and MCOs about the identification of “unreachable” Medicaid members eligible for preventive health care. All four of the components of the computer program product are connected with each other using internet connections.

### BRIEF DESCRIPTION OF THE DRAWINGS

FIG. 1. Landscape of member outreach platforms. Shown in this illustration are the key players in the area of identifying “unreachable” Medicaid beneficiaries. The key players have been placed in a two-dimensional matrix. In one dimension, this matrix scores the players for the level of sophistication in their operation. For example, if a player shown in this matrix is using a software program for identifying “unreachable” Medicaid beneficiaries, that player is referred as a high-tech player and is scored at the top of the unit-less Y-axis. Since this player is not physically reaching out to the “unreachable” Medicaid beneficiary, this player is placed at the lower end of the unit-less X-axis. On the other hand, if a player is physically reaching out to an “unreachable” Medicaid beneficiary without the use of any software program, that player is referred is high-touch player and is scored at the right-end of the unit-less X-axis and the bottom of the unit-less Y-axis. In this graphical illustration, bosWell, the digital health software application of the present invention, is scored at the top of the unit-less Y-axis and the right-end of the unit-less X-axis.

FIG. 2. Functional organization of the Medicaid value chain. State Medicaid programs sit at the top of the Medicaid value chain. State Medicaid programs receive funding from local taxpayer dollars as well matching funds from the federal government and distribute to ACOs and MCOs which in turn cover the costs of visits to Federally Qualified Health Centers (FQHCs) that serve as touch points for primary, and preventive care for Medicaid members labeled as “Patients” in this illustration. Community Based Organizations (CBOs) are labeled here as “users” of bosWell, the digital health software platform of the present invention, used to identify “unreachable” Medicaid beneficiaries. ACOs and MCOs are the ultimate customers that subscribe to the bosWell platform in order to better locate “unreachable” members and facilitate their engagement in preventive health care. According to the present invention, FQHCs serve as a channel to onboard both CBOs as well as ACOs and MCOs onto the bosWel platform.

FIG. 3. Properties associated with bosWell, the software platform of the present invention. The bosWell platform provides free access to a HIPPA-complaint web-based application and allows CBOs to generate customizable intake forms to build longitudinal profiles of the clients served for the purpose of tracking program performance and effectively reporting data.

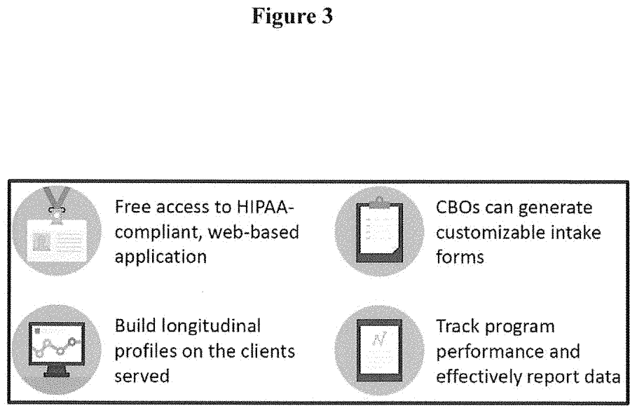

FIG. 4. A block flow diagram of a computer-based system according to the present invention for connecting “unreachable” Medicaid members to preventive health care by their respective care managers at ACOs and MCOs.

FIG. 5. Member matching and notification component of bosWell, the platform of the present invention. The front end application used by CBOs generates longitudinal personal data on Medicaid beneficiaries visiting CBOs and the matching engine embedded in the software platform queries this information against ACO and MCO enrollment files to locate “unreachable” members while establishing a channel for engagement.

FIG. 6. Hub and Spoke Care Model of bosWell. The applicant has built an end-to-end solution, bosWell, the digital health care platform of the present invention. Using bosWell, the Applicant has built an end-to-end solution that strengthens safety-net community-based organizations (CBOs) while extending the reach of ACO and MCO care management teams, which represent a member's “medical home”, into the neighborhoods where Medicaid members reside.

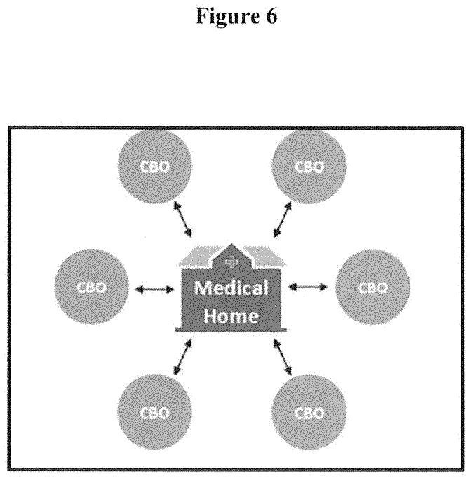

### DETAILED DESCRIPTION OF THE PREFERRED EMBODIMENTS

Reference is made herein in detail to specific embodiments of the invention. Specific examples are illustrated with drawings. The subject matters of embodiments of the present invention are provided herein to satisfy the statutory requirement. However, the description provided herein is not meant to limit the scope of the present invention. Rather the claimed subject matter of the present invention may be embodied in several other ways within the scope of the present invention.

The present invention provides a novel digital health care platform, bosWell, to reach Medicaid members and facilitate their engagement in preventive care. The following definitions are provided herein to describe the method for identifying and connecting unengaged Medicaid members to preventive health care through channels to their respective ACO and MCO care management teams that function as “medical homes” according to the present invention.

The term “super-utilizer” as used in this invention means a Medicaid member who is utilizing costly emergency medical services while skipping primary care facilities and preventive health care measures.

The term “unreachable” is used to refer to Medicaid members who are reported as missing by their respective ACOs and MCO due to outdated contact information as well as a lack of engagement in preventive care.

FQHCs are a means to provide preventive health care to Medicaid members. Over 1,400 FQHCs with more than 10,000 sites exist across United States and serve approximately 25 million patients annually. The present invention envisions each FQHC as a node around which to build a network of CBOs using bosWell, the digital health platform to identify and engage “unreachable” Medicaid members in preventive health care.

Community-based organizations (CBOs) are shelters, food pantries, resource centers, mobile health care clinics and other touch points that Medicaid members rely on for their, food, clothing, health care and hygiene needs. Based on market surveys, it is estimated that more than 400,000 CBOs exist across the United States comprising faith-based organizations, food pantries, mobile clinics, shelters, resource centers and day programs.

Primary health care refers to an annual health examination to assure that an individual is in good condition and attending to any immediate medical needs required for the well-being of the beneficiary.

Preventive health care means treatments including periodic diagnosis, medication, and use of medical devices to control any health condition which left untreated would damage one or another part of the body leading to physical and mental disability.

Enrollment data means the data available through ACOs and MCOs about the Medicaid population that is to be served with the funding received from State Medicaid Programs.

The term “bosWell” as used in this invention refers to the digital health platform of the present invention useful in identifying “unreachable” Medicaid beneficiaries.

Client management application refers to a software program used at the front-end of bosWell. This software program is embedded in the computer devices of CBOs to record client data.

Matching engine, also referred to as matching machine, is a software program functioning at the back-end of bosWell to identify “unreachable” Medicaid members.

The terms “Medicaid beneficiary”, “patient”, and “Medicaid patient” as well as the plural forms of these terms are used interchangeably and refer to individuals receiving health care coverage through Medicaid.

Without updated contact and clinical information on “unreachable” members, ACOs and MCOs lack a baseline to effectively deliver care. From a compliance standpoint, State Medicaid agencies impose varying degrees of financial penalties for incomplete health risk assessments (HRAs) and missing targets for preventive screening/wellness visits. Non-compliance with State Medicaid guidelines not only affects ACO/MCO quality ratings, but it can also lead to reduced market share of covered members and ultimately the inability to renew contracts.

ACOs and MCOs are also particularly concerned about the costs associated with members who are unengaged in preventive care. More often than not, “unreachables” are discovered through subsequent visits to hospital emergency departments and categorized as “super-utilizers” through retrospective analyses of claims data. In order to curtail this phenomenon, ACOs and MCOs invest heavily in outreach initiatives to ensure that their members access earlier, preventive interventions. These engagement strategies include non-emergency medical transportation (NEMT) to help members access clinics/pharmacies, telehealth platforms to provide virtual consultations and coaching programs to boost adherence to treatment regiments.

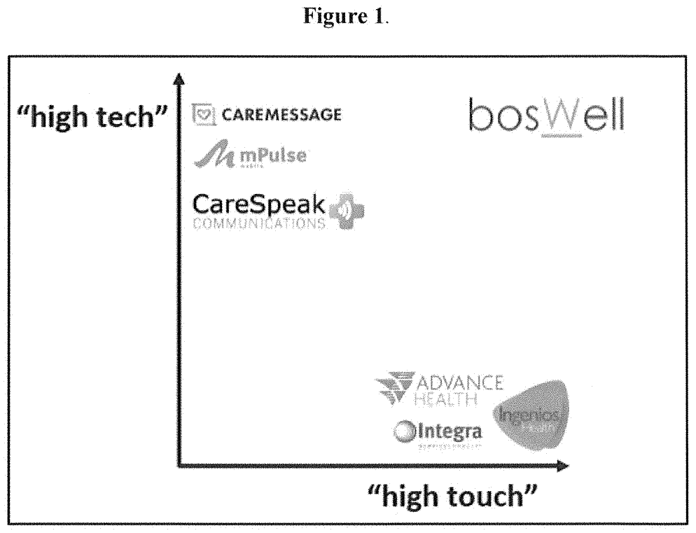

Studies have shown both clinical and financial benefits of care management for vulnerable Medicaid members. Such initiatives have been proven to reduce emergency department utilization by up to four-fold, while creating a 44% return on investment. Timing of these interventions, however, is critical given the fact that two-thirds to three-quarters of “super-utilizer” populations turnover each year—constantly being replaced by the next wave of hospital “frequent fliers.” Missing out on cost-effective opportunities to identify individuals with the greatest needs and deliver care in a preventive manner perpetuates a vicious cycle that is costly to both health care organizations and Medicaid members. Predominant strategies for member outreach are based on “high tech” and “high touch” methods (FIG. 1).

Companies in the “high tech” realm include CareMessage, mPulse Mobile and CareSpeak Communications. These organizations have software platforms to connect health care organizations to their Medicaid members through mobile engagement consisting of automated text messages such as reminders for prescription refills and appointments. While these “high tech” approaches certainly improve the bandwidth of health care organizations, they do not always reach Medicaid members. Lower-income populations are prone to have greater inconsistencies with connectivity and a tendency to frequently change mobile phone numbers, thereby eliminating the channel for engagement.

The “high touch” models, also known as “feet on the street,” consist of hiring teams of individuals to conduct outreach and track down “unreachable” Medicaid members. Integra Services Connect, Advance Health and Ingenios are among the leaders in this space. In addition to navigators at ACOs and MCOs, these companies also send their staff into the community—earning revenue per member located and engaged. The costs of hiring navigators and outreach workers makes this model difficult to scale, particularly with regards to the transient nature of the Medicaid population. Given the challenges of locating “unreachable” members through conventional door-knocking and neighborhood canvassing, the “high touch” models have shifted towards addressing the needs of elderly Medicare members in home-based settings.

The market seeks a scalable as well as sustainable solution to reliably locate and engage “unreachable” Medicaid members. Through the development of bosWell, the digital health platform of the present invention, the applicant addresses these needs while producing a unique platform that is based on “high tech enabling high touch.

The front-end, client management application software of the present invention scales effectively since community-based organizations (CBOs) such as shelters, food pantries, resource centers and mobile clinics readily adopt free tools to improve organizational bandwidth as well as data reporting. CBOs are predominantly non-profits with limited resources and currently utilize pen/paper or homegrown databases for client management. This leads to workflow redundancies and the potential to compromise finding due to data under-reporting.

Staff using bosWell, the digital health software platform of the present invention, at the CBO level have strong, longitudinal relationships with their clients and serve as critical liaisons to link Medicaid members to their respective care management teams on the back-end. ACOs and MCOs subscribing to bosWell, the digital health software platform of the present invention, will receive alerts when specific members check into CBOs, then have the opportunity to push information back to staff at the frontline.

FQHCs operate as primary care practices for the Medicaid population and receive enhanced reimbursement rates from the Health Resources and Services Administration (HRSA). ACOs incorporate FQHCs into their networks, offering value-based financial incentives for meeting population health benchmarks. Similarly, FQHCs are essential vehicles for MCOs to deliver primary care to their members and function on volume-based revenue models.

Over 1,400 FQHCs with more than 10,000 sites exist across the country and serve approximately 25 million patients annually. The applicant sees each site as a node around which to build a network of CBOs using bosWell, the digital health software platform of the present invention, particularly given the intimate knowledge FQHCs have with community stakeholders.

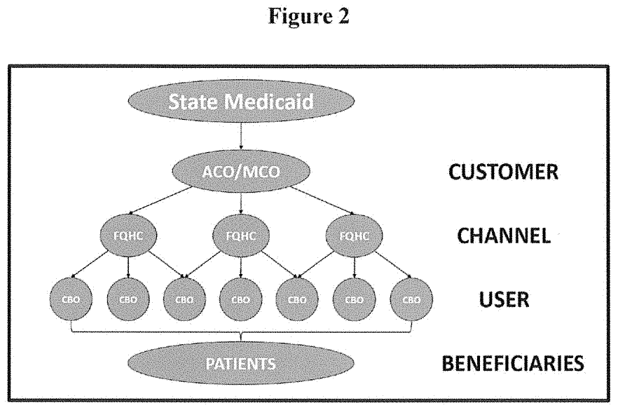

FIG. 2 illustrates interactions among the various players within the Medicaid program. State Medicaid Programs in each state control the respective budgets for Medicaid. State Medicaid Programs enter into contractual relationships with ACOs and MCOs. The ACOs and MCOs receive capitated payment as well as value-based incentives from State Medicaid Programs to provide health care coverage to assigned Medicaid members and to ensure access to preventive health care, often through primary care FQHC providers. Thus, a Medicaid member living in a particular geographical region has a designated ACO or MCO to pay for his/her health care from State Medicaid funds and also a dedicated FQHC that serves as a touch point for accessing primary care. FQHCs get reimbursed by ACOs and MCOs for the services rendered to a Medicaid member. The method according to the present invention to identify and engage “unreachable” Medicaid members in preventive health care brings CBOs into the functional delivery of services to Medicaid beneficiaries and makes CBOs an integral component in extending the capacity of care management teams at ACOs and MCOs to serve their members.

The method according to the present invention to identify and engage “unreachable” Medicaid members in preventive health care involves the use of bosWell, a computer-based digital health care platform of the present invention. The front end of bosWell is a client management application and it is distributed to CBOs. Traditionally, the CBO are accustomed to use the paper and pencil to collect information about the people visiting them. Staff at the CBO, with the digital client management software will be able to collect data about their client populations in a very efficient way and be able to establish longitudinal relationships with their clients and serve as critical liaisons to link Medicaid members to their respective care management teams on the back-end of bosWell.

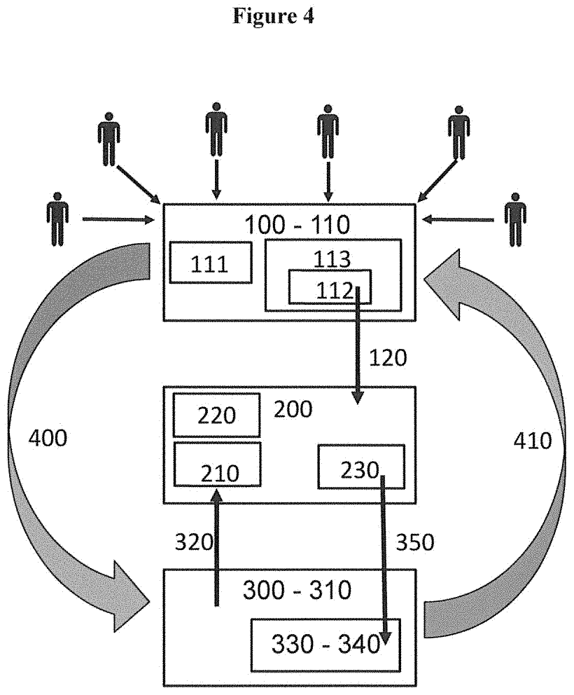

FIG. 4 illustrates the role of bosWell in managing the health care of the Medicaid population with the input from ACOs, MCOs and FQHCs. In the FIG. 4, the plurality of CBOs 100-110 are equipped with the client management software 111, functioning at the front end of boswell, the digital health care platform of the present invention. Each CBO possessing the client management software 111 has the ability to collect and store personal data in the digital form along with the ability to analyze the longitudinal data on the Medicaid population it serves. When the clients visit a CBO to address any of their needs, the staff at the CBO collect personal information and builds a profile for each client. Before disclosing any data on their clients, the staff at the CBOs obtain the consent of the clients in accordance with the state and federal health care compliance requirements. More importantly, in order to protect the privacy of the clients, the staff at the CBOs will strictly follow the requirements under Health Insurance Portability and Accountability Act of 1996 (HIPAA). The entire bosWell application is built according to HIPPA-complaint standards using a secure web hosting platform that includes risk assessments, policies and procedures, incident response as well as application security management configuration. Clients opting into bosWell at the CBO level will complete an electronic consent form, allowing for their demographic information to be queried against available health care organization enrollment files. The standard consent form requests the client's name (FN, MI/N, LN), sex, DOB and ZIP. The staff at the CBOs will collect required information 112 using the client management software application 111 embedded in a desktop computer, laptop computer, a tablet or a mobile device.

The data collected at CBOs 112 using the client management software application 111 will be stored in a secure and HIPAA-compliant web server 113 before being transferred to matching engine 200 at the back end of bosWell, the digital health care platform of the present invention. The matching engine 200 is housed within a separate, dedicated server and receives the data 112 collected at the CBOs 100-110 through an internet connection 120.

Stored within the matching engine 200 are the Medicaid member enrollment data files 210 received from plurality of ACOs and MCOs 300-310. Using the connection established through bosWell, the plurality of ACOs and MCOs 300-310 transfer their Medicaid member enrollment data files 210 to the matching engine 200 using a secured connection 320. In one aspect of the present invention, the matching engine using software 220 performs simple deterministic “look up” functions on first name, last name, gender as well as date of birth between the CBO data 112 and member enrollment file 210. In another aspect of the present invention, in order to improve the sensitivity and specificity of the matching process, a rule-based matching system using a combination of patient trait is adopted.

Following the initial linkage of CBO data 112 to matching engine 200, bosWell matching software application 220 embedded within the matching engine 200 will query the CBO data 112 against the client enrollment data 210 to identify the data related to “unreachable” Medicaid beneficiaries 230. In collecting the data related to “unreachable” Medicaid beneficiaries 230, the bosWell matching software application 220 takes into account the following information: (1) Member demographic information; (2) Current member contact information, if collected by CBO, and date of collection; (3). Level of the match; and (4) Uniqueness of the match. Once an “unreachable” Medicaid beneficiary 230 is identified, matching engine 200 sends out an alert to the care management team 330-340 at the plurality of ACOs and MCOs using a secured internet connection 350.

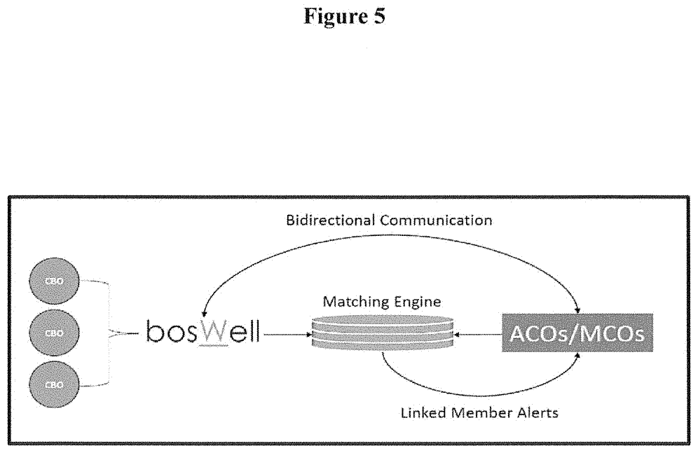

The bidirectional communications 400 and 410 between care management teams at ACOs and MCOs 330-340 and CBOs 100-110 allow for the former to guide decision making for the latter. Subsequently, CBOs 100-110 can assist ACOs and MCOs 300-310 to better monitor their newly linked members in real-time. This type of interaction extends the capacity of care managers beyond the conventional medical home to include CBOs, thereby creating a hub and spoke model (FIG. 5).

In one aspect of the bosWell hub and spoke model, a health risk assessment will be embedded within the regular client management software application 111. Health risk assessments are critical for ACOs and MCOs to obtain a baseline on their patient population and to receive adequate resources from the state Medicaid agencies. Health risk assessments are generally mailed to members, administered telephonically or completed during primary care visits—the reasons why “unreachable” Medicaid members have low completion rates. Maintaining the health risk assessments at the CBO level is expected to improve the health care delivery to the Medicaid population, particularly to those designated as “unreachable” Medicaid members.

In another aspect of the bosWell hub and spoke model, the technology-enabled CBO will have the opportunity to connect the identified “unreachable” Medicaid member to care management teams at their respective medical home through the readily available telehealth tools. The staff members at the CBO could arrange for a teleconference or a video conference between the Medicaid member and the care management teams at ACOs and MCOs. The staff member at the CBO, through guidance from care managers at ACOs and MCOs, could also arrange for non-emergency transportation of the Medicaid member to his/her FQHC for preventive health care treatment.

As explained above, the present invention has built an end-to-end solution using bosWell, the digital health platform of the present invention that strengthens safety-net community-based organizations (CBOs) while extending the reach of care management teams at Medicaid ACOs and MCOs into the neighborhoods where Medicaid members reside. Over the past two years, the front-end client management application has undergone extensive feature development to improve user experience/interface (UX/UI) and meet the specific needs of CBOs. In order to ensure secure transmission and storage of client information, bosWell is hosted according to The Health Insurance Portability and Accountability Act of 1996 (HIPAA) guidelines.

Another important aspect of the present invention is that bosWell, the digital health platform of the present invention, leverages a trust spectrum in health care to more effectively engage Medicaid members. Given the dependence of vulnerable populations on CBOs as survival touch points, it is believed that CBO staff have stronger relationships with Medicaid members than any other stakeholder in the health care system. Data from the existing bosWell CBO network suggests that clients visit a given touch point an average of 20 times per year, which also indicates potential for multiple engagement opportunities.

### REFERENCES

U.S. Pat. No. 6,044,351

U.S. Pat. No. 7,158,937

U.S. Pat. No. 7,801,740

U.S. Pat. No. 8,355,926

U.S. Pat. No. 8,528,056

U.S. Pat. No. 9,589,244

U.S. Pat. No. 10,121,557

U.S. Patent Application Publication No. 2017/0124282

Baer A., and M. Baker (2017). Reducing Barriers to Medication Access and Adherence for ACA and Medicaid Participants: A Peer-to-Peer, Community-Based Approach. Am J Manag. Care. 23(3 Suppl): S54-S57.

Barth S. and B. Ensslin (2014) Center for Health Care Strategies, Inc. Contacting Hard-to-Locate Medicare and Medicaid Members: Tips for Health Plans. https://www.chcs.org/media/PRIDE-Tips-for-Contacting-Hard-to-Locate-Members_121014_2.pdf Published December 2014.

Beck, A. F., K. L. Anderson, K. Rich, S. C. Taylor, S. B. Iyer, U. R. Kotagal and R. S. Kahn (2019) Cooling the hot spots where child hospitalization rates are high: A neighborhood approach to population health. Heath Affairs 38(9): 1433-1441.

Bleser, W. K., R. S. Saunders, L. Winfield, Japinga, M. Smith, N, Kaufman, B. G., Crook, H. L. Muhlestein, D. B. and M. McClellan (2019) ACO serious illness care: Survey and case studies depict current challenges and future opportunities. Health Affairs 38(6): 1011-1020.

Brailer, D. J. (2010) Guiding the health information technology agenda. Health Affairs 29(4): 586-595.

Bylander, J. (2019) Community-focused health care for the seriously ill. Health Affairs 38 (3): 344-346.

Cunningham, R. (2015) Once a welfare add-on, Medicaid takes charge in reinventing care. Medical Affairs 34(7): 1080-1083.

Cunningham, P. J. (2015) Many Medicaid beneficiaries receive care consistent with attributed of patient-centered medical home. Medical Affairs 34(7): 1105-1111.

Davies M L, Goffman R M, May J H, et al. Large-Scale No-Show Patterns and Distributions for Clinic Operational Research. Healthcare. 2016; 4(15): 1-12.

Donelan, K., Y. Chang, J. Berrett-Abebe, J. Spetz, D. I. Auerbach, L. Norman and P. I. Buerhaus (2019) Care management for older adults: The roles of nurses, social workers, and physicians. Health Affairs 38(6): 941-949.

Fisher, E. S., S. M. Shortell, S. A. Kreindler, A. D. van Citters and B. K. Larson (2012) A framework for evaluating the formation, implementation, and performance of accountable care organizations. Health Affairs 31(11): 2368-2378.

Fraze, T. K., Belder, L. B., Briggs. A. D. M. and Colla, C. H. (2019) Eyes in the home: ACOs use home visits to improve care management, identify need, and reduce hospital use. Health Affairs 38(6): 1021-1027.

Graig, L., S. M. Cruz and J. Alper (2018) Implementing quality measures for accountability in community-based care for people with serious illness. Proceedings of a Workshop. The national Academies Press. Washington D.C.

Gryczynski, J., R. P. Schwartz, K. E. O'Grady, L. Restivo, S. G. Mitchell and J. H. Jaffe (2016) Understanding patters of high-cost health care use across different substance user groups. Health Affairs 35(1): 12-19.

Grover C A, Close R J H, Villareal K, Goldman L M. Emergency Department Frequent User: Pilot Study of Intensive Case Management to Reduce Visits and Computed Tomography. West J Emerg Med. 2010; 11(4): 336-343.

Gusmano, M. K. and F. J. Thompson (2015) An examination of Medicaid delivery system reform incentive payment initiatives under way in six states. Health Affairs 34(7): 1162-1169.

Henry J. Kaiser Family Foundation. Medicaid Enrollment & Spending Growth: FY 2017 & 2018. https://www.kff.org/medicaid/issue-brief/medicaid-enrollment-spending-growth-fy-2017-2018.

Halamaka, J. D. (2010) Making the most of federal health information technology regulations. Health Affairs 29(4): 596-600.

Johnson T L, Rinehart D J, Durfee J, et al. For many patients who use large amounts of health care services, the need is intense yet temporary. Health Aff. 2015; 34(8): 1312-19.

Lipika, S., H. E. Hutton, E. J. Erbelding, et al. (2010) Digital divide: variation in interne and cellular phone use among women attending an urban sexually transmitted infections clinic. J Urban Health. 87(1):122-128.

Long, P., M. Abrams, A. Milstein, G. Anderson, K. L. Apton. M. L. Dahlberg and D. Whicher (2017) Effective care for high-need patients. National Acadmey of Medicine, Washington D.C.

Mayben, J. K. and T. P. Giordano (2007). Internet use among low-income persons recently diagnosed with HIV infection. AIDS Care. 19(9):1182-1187.

Medicaid and CHIP Payment and Access Commission. Medicaid Payment Policy for Federally Qualified Health Centers. https://www.macpac.gov/wp-content/uploads/2017/12/Medicaid-Payment-Policy-for-Federally-Qualified-Health-Centers.pdf Published December 2017.

Medicaid.gov, Medicaid & CHIP Enrollment Data Highlights; March 2018 Medicaid and CHIP Enrollment Data Highlights. https://www.medicaid.gov/medicaid/program-information/medicaid-and-chip-enrollment-data/report-highlights/index.html

Meyer, H. (2011) Accountable care organization prototypes: winners and losers? Health Affairs 30(7): 1227-1231.

Meyer, H. (2012) Many accountable care organizations are now up and running, it not off to the races. Health Affairs 31(11): 2363-2367.

Musumeci M B, Rudowitz R. Henry J. Kaiser Family Foundation. Medicaid Non-Emergency Medical Transportation: Overview and Key Issues in Medicaid Expansion Waivers. https://www.kff.org/medicaid/issue-brief/medicaid-non-emergency-medical-transportation-overview-and-key-issues-in-Medicaid-expansion-waivers.

Nutting, P. A., Crabtree, B. F. and McDaniel, R. R. (2012) Small primary care practices face four hurdles—including a physician-centric mind-set—In becoming medical homes. Health Affairs 31(11): 2417-2422.

Oliveira, C., J. Cheng, S. Vigod, J. Rehm and P. Kurdyak (2016) Patients with high mental health costs incur over 30 percent more costs than other high-cost patients. Health Affairs 35(1): 36-43.

Oung, A. B., E. Kosirog, B. Chavez, J. Brunner and J. J. Saseen (2017) Evaluation of medication adherence in chronic disease at a federally qualified health center. Therapeutic Advances in Chronic Disease. 8: 113-120.

(Pourat, N., A. C. Davis, X. Chen, S. Vrungos and G. F. kominski (2015) In California, primary care continuity was associated with reduced emergency department use and fewer hospitalization. Health Affairs 34(7) 1113-1120.

Rumball-Smith, J. (2017) Using the electronic health record to understand and minimize overuse. JAMA 317(3): 257.

Schorsch K. Crain's Chicago Business. Medicaid's M.I.A. Health plans struggle to find their enrollee. http://www.chicagobusiness.com/article/20170708/ISSUE01/170709929/the-problem-in-illinois-no-one-is-talking-about.

Severin, C. (2017) Community Care Cooperative. Creating an FQHC-based Medicaid Accountable Care Organization. http://www.healthcarevaluehub.org/files/5515/1058/4958/Severin.pdf Published November 2017.

Shin, P., J. Sharac and S. Rosenblum (2015) Community health centers and Medicaid at 50: An enduring relationship essential for health system transformation. Health Affairs 34(7): 1096-1104.

Shrank, W. H., T. L. Rogstad and N. Parekh (2019) Waste in the US health care system. Estimated costs and potential savings. JAMA 322(15): 1501-1509.

Shumway M., A. Boccellari, K. Brien and R. L. Okin (2008) Cost-effectiveness of clinical case management for ED frequent users: results of a randomized trial. Am J Emerg Med. 26(2): 155-164.

Sparer, M. S. (2015) Medicaid at 50: Remarkable growth fueled by unexpected politics. Health Affairs 34(7): 1084-1091.

Spetz, J., R. L. Stone, S. A. Chapman and N. Bryant (2019) Home and community-based workforce for patients with serious illness requires support to meet growing needs. Health Affairs 38 (6): 902-909.

Stamy C D, MacKinney A C. Center for Rural Health Policy Analysis. Emergency Department Super Utilizer Programs. https://cph.uiowa.edu/ruralhealthvalue/files/Super%20Utilizers.pdf Published October 2013.

Stanton M W, Rutherford M K. Agency for Healthcare Research and Quality. The high concentration of US health care expenditures. http://meps.ahrq.gov/mepsweb/data_files/publications/ra19/ra19.pdf Published June 2006.

Tai-Seale, M., N. Lance-Downing, V. G. Jones, R. V. Milani, B. Zhao, B. Clay, C. D. sharp, A. S. Chan and C. A. Longhurst (2019) Technology-enabled consumer engagement: promising practices at four health care delivery organizations. Health Affairs 38 (3): 383-390.

Takach, M. (2012) About half of the states are implementing patient-centered medical homes for their Medicaid populations. Health Affairs 31(11): 2432-2440.

The Sequoia Project. A Framework for Cross-Organizational Patient Identity Management. https://sequoiaproject.org/wp-content/uploads/2015/11/The-Sequoia-Project-Framework-for-Patient-Identity-Management.pdf Published November 2015.

Vaida, B. L. (2019) For the uninsured in Memphis, a stronger safety net. Health Affairs 38(9): 1420-1424.

Vulimiri, M., W. K. Bleser, R. S. Saunders, F. Madanay, C. Moseley, F. H. Mcguire, P. A. Ubel, A. McKethan, M. McClellan and C. A. Wong. (2019) Engaging beneficiaries in Medicaid programs that incentivize health promoting behaviors. Health Affairs 38 (3) 431-439.

Weil, A. R. (2015) Medicaid moving forward. Health Affairs 34(7): 1092-1095.

Weil, A. R. (2019) High-cost populations, the ACA, and more. Health Affairs 38(6):7.

Weil, A. R. (2019) Community care for high-need patients. Health Affairs 38(6): 891.

Weil, A. R. (2019) Neighborhoods and health, Medicaid, and more. Health Affairs 38(9) 1419.

Wicklund, E. Health Intelligence. State Medicaid Programs Are Seeing the Value of Telehealth at Home. https://mhealthintelligence.com/news/state-medicaid-programs-are-seeing-the-value-of-telehealth-at-home.

Wright, B. J., K. B. Vartanian, H-F. Li, N. Royal and J>K. Matson (2016) Formerly homeless people had lower overall health care expenditure after moving into supportive housing. Health Affairs 35(1): 20-27.

What is claimed:

1. A method for connecting unreachable Medicaid members to preventive health care in their respective medical home, comprising:
(a) providing community-based organizations with client management software to collect and digitize data related to Medicaid members visiting community-based organizations;
(b) training staff at community-based organizations in using the client management software and maintaining compliance requirements for collecting personal data;
(c) obtaining consent from Medicaid members visiting community-based organizations for collecting and sharing their personal data;
(d) collecting personal data from the Medicaid members visiting community-based organizations and transferring to a matching engine comprising enrollment data from Medicaid accountable and managed care organizations;
(e) querying personal data collected at community-based organizations with enrollment data from Medicaid accountable and managed care organizations stored in the matching engine and identifying “unreachable” Medicaid members; and
(f) notifying said Medicaid accountable and managed care organizations about the identification of unreachable Medicaid members.

2. The method for connecting unreachable Medicaid members to preventive health care in claim 1, wherein said respective medical home the care management team at the accountable or managed care organization to which the member is attributed.

3. The method for connecting unreachable Medicaid members to preventive health care in claim 1, wherein said community-based organization is selected from a group consisting of shelters, food pantries, resource centers, mobile clinics and touch points for survival.

4. The method for connecting unreachable Medicaid members to preventive health care in claim 1, wherein said compliance requirement for collecting personal healthcare data is in accordance with the guidelines under the Health Insurance Portability and Accountability Act of 1996.

5. The method for connecting unreachable Medicaid members to preventive health care in claim 1, wherein said client management tool is modular and allows community-based organizations to customize intake forms to specific reporting needs.

6. The method for connecting unreachable Medicaid members to preventive health care in claim 1, wherein said personal data comprise longitudinal profile of the Medicaid members served

7. The method for connecting unreachable Medicaid members to preventive health care in claim 1, wherein said query of the personal data collected at community-based organizations with the enrollment data from Medicaid accountable and managed care organizations stored in the matching engine to identify unreachable Medicaid members is performed by using simple deterministic look up functions based on first name, last name, gender and date of birth.

8. The method for connecting unreachable Medicaid members to preventive health care in claim 1, wherein said query of the personal data collected at community-based organizations with enrollment data from Medicaid accountable and managed care organizations stored in the matching engine to identify unreachable Medicaid members is performed by using rules-based matching algorithms.

9. The method for connecting unreachable Medicaid members to preventive health care in claim 1, and further comprising a step of continuously updating the enrollment data from Medicaid accountable and managed care organizations stored in the matching engine.

10. The method for connecting unreachable Medicaid members to preventive health care in claim 1, and further comprising a step of periodically updating the enrollment data from Medicaid accountable and managed care organizations stored in the matching engine.

11. The method for connecting unreachable Medicaid members to preventive health care in claim 1, and further comprising a step of engaging “unreachable” Medicaid members with care management teams via staff at community-based organizations.

12. The method for connecting unreachable Medicaid members to preventive health care in claim 1, and further comprising a step of providing “unreachable” Medicaid members with non-emergency medical transportation to receive preventive health care.

13. The method for connecting “unreachable” Medicaid members to preventive health care in claim 1, and further comprising a step of offering virtual telehealth consultations such as the “unreachable” Medicaid members may engage with primary care from community-based organizations.

14. A system for connecting unreachable Medicaid members to preventive health care in their respective medical home, comprising:
(a) a plurality of electronic devices distributed at a plurality of community-based organizations for capturing longitudinal profiles of personal records of Medicaid members visiting said community based organizations in an electronic format;
(b) a repository of enrollment data from Medicaid accountable and managed care organizations;
(c) a matching engine to query the longitudinal personal data captured at community-based organizations with enrollment data from Medicaid accountable and managed care organizations stored in the repository for the purpose of identifying unreachable Medicaid members; and
(d) an electronic means for notifying said Medicaid accountable and managed care organizations about the identification of the unreachable Medicaid members eligible for preventive health care.

15. A computer program product embodied on a computer readable storage medium for processing digital health records to identify unreachable Medicaid members comprising:
(a) a computer code for receiving personal records of Medicaid members visiting community-based organizations and generating a longitudinal electronic personal record of said Medicaid members;
(b) a computer code for receiving and storing electronic enrollment record of Medicaid members from accountable and managed care organizations in a central repository;
(c) an algorithm for querying the electronic personal records in step (a) with electronic enrollment records or electronic claim record in step (b) for the purpose of identifying unreachable Medicaid members; and
(d) an electronic means for alerting said accountable and managed care organizations about the identification of unreachable Medicaid members eligible for preventive health care.
